(19) United States
(12) Patent Application Publication
(10) Pub. No.: US 2009/0026642 A1
(43) Pub. Date: Jan. 29, 2009

(54) Stone cast

(76) Inventor: Dennis Joseph White, Plainsboro, NJ (US)

Correspondence Address: Dennis J. White, 41 North Main Street, Cranbury, NJ, 08512, US

(21) Appl. No.: 12/006,379

(22) Filed: Jan. 2, 2008

Related U.S. Application Data
(63) Continuation-in-part of application No. 11/878,337, filed on Jul. 23, 2007.

(51) Int. Cl. A61F 2/00; B29C 39/02; B29D 31/00; A61C 13/20

(52) U.S. Cl. 264/16; 264/308

(57) ABSTRACT

A method is disclosed for forming an accurate stone cast from an impression of a body region, used for the purpose of prosthetic fabrication, such as dental crowns and artificial eyes and hearing aids. The initial step of this unique method is to affix a relatively thin mix of wet dental stone in contact with the internal surface of the impression. After this initial layering has cured, a subsequent pour is made to sufficiently fill the impression to form a cast.

### CROSS REFERENCE TO RELATED APPLICATIONS

This application is a Continuation-In-Part related to application Ser. No., 11/878,337, filed Jul. 23, 2007, title: METHOD AND MATERIAL TO FORM A CAST FROM GYPSUM

### FIELD OF INVENTION

This invention relates generally to making better fitting dental restorations and more specifically this invention relates to making accurate stone dental casts and models, which are used in the process of fabricating removable dental appliances and indirect dental restorations.

### DESCRIPTION OF PRIOR ART

Stone dental casts need be very accurate replicas of the dentition. The cast, along with other exact measurements and guidelines supplied by the dentist, enables the laboratory to make dentures, partial dentures, orthodontic appliances, bleaching trays, and nightguards. Other special applications, such as oral snoring devices, obturators, and surgical stents, may also be prescribed by the dentist.

Once a cast is made, it can be sectioned so that segments can be removed. Segments representing individual teeth are referred to as dies. The dies are indexed in various ways to give an accurate and reliable placement of the removable die in and out of the model base.

Some practitioners will refer to this dental cast with removable dies as a dental model. And they will refer to the one piece stone pour of the impression as a dental cast.

However, many practitioners will still use the term “cast” and “model” interchangeably and do not distinguish between the two terms.

Dental models are used in the dental laboratory to create dental restorations which ultimately will be delivered to the dentist office. These restorations are then affixed into, or onto, the patient's tooth with a cement, such as zinc phosphate cement. These cemented restorations are referred to as indirect restorations. Examples of indirect restorations would include inlays, onlays, veneers and crowns.

Conversely, direct restorations are those restorations that are buildup directly, and usually incrementally, into the patients tooth. Silver amalgams and composites are examples of direct dental restorations.

When initiating an indirect restoration for a patient, the dentist will first carve the tooth structure into a certain shape. This finished carved tooth shape is referred to as a prepped tooth, or simply, the preparation. After this step of tooth preparation is accomplished, impressions are taken to record the new altered dimensions, that is, a moldable material is impressed upon the tooth and allowed to set. Elastomeric materials are sometimes employed for this. Examples of elastomeric impression materials would include polyvinylsiloxane, polyether, and rubber base.

Another group of impression materials, called hydrocolloid, contain agar and water. Irreversible hydrocolloid is made by addition of water to a dry alginate formula. The resultant gel is in a moldable state for a given period of time. While it is in this moldable state it is impressed onto the mouth structures and held immobilized until it sets.

Another agar containing impression material is reversible hydrocolloid. Reversible hydrocolloid is brought to a predetermined elevated temperature to form a gel. While the hydrocolloid is in this warmed gel state it is impressed to the oral structures. This reversible hydrocolloid is then cooled to final set in the mouth by using water cooled metal trays.

Collectively, all of the impression materials cure to the shape of the teeth and mouth. The impression material is removed from the mouth, capturing a negative image of the impressed area of the oral cavity.

In the lab, a wet mixture of stone is poured into the impression. Upon hardening, after an hour or so, the resultant dental cast is separated from the impression. This cast will be used as a mold in the process of fabricating removable appliances, such as dentures and the like.

The fabricated dentures are delivered to the patient by the dentist. Any irregularities in the fit of the appliance are located and adjusted by the dentist. A similar process takes place for all removable dental appliances.

Crowns are also delivered from the dental laboratory to the dentist office. These restorations are then modified and cemented for the patient by the practitioner. The crown must be adjusted in accordance to three parameters. First, it must fit the tooth to which it is cemented to. It must also be adjusted to the adjacent teeth to yield a certain tug feel with dental floss. Last, it must accommodate the opposing tooth cuspal relation so that the new crown is not high and does not strike the opposite tooth when the patient closes.

So the process of taking impressions avail the patient dentures and cast crowns. These products would be very challenging to make without an indirect technique.

And making stone casts from impressions is not exclusive to dentistry.

Similar to the process of dental appliance fabrication, is the process of artificial eye fabrication. The process of eye reconstruction is basically borrowed from the dental field. Dental impression materials are utilized directly or sometimes used with a modified viscosity to impress areas of the eye. The impressions are poured with gypsum products. In the most common technique, the sculpted wax pattern for the artificial eye is flasked for final process, http://artificialeyeclinic.com/fabricating_prosthesis.html. This technique is very similar to techniques employed by a dental technician, who flasks a sculpted waxed denture.

And similar again to the above indirect processes, is the replication of the ear canal and related surrounding structures for the fabrication of hearing aids.

Impressions are taken for many specific prosthetic applications in form and function for other areas of the body.

### OBJECTS AND ADVANTAGES

A tremendous problem exists with indirect dentistry. Dental appliances and indirect restorations seldom have a perfect fit. Most mouth appliances, such as dentures, and all tooth restorations, such as crowns, need varying amounts of chair time to adjust. Distortions must be identified and compensated for, before finalizing the products. In regard to acrylic appliances, professionals believe distortion may occur during the setting phase of the acrylic. Indeed, dentures do require multiple appointments to finalize. And usually, the final outcome is a slight disappointment. Dentures are usually tolerated by the wearer; dentures are seldom as tight fitting as the dentist would prefer.

Similar to the irregularities associated with dentures, acrylic nightguards also usually end up with a compromised outcome. It is very time consuming to troubleshoot an ill-fitting nightguard, in fact, many dentists have resorted to a soft inside liner attached to the hard outside acrylic shell. This laminate design is not so much for patient comfort, but for ease of delivery. The soft material is very moldable and therefore does not have to be a precise fit to the dentition. Its inherent conforming shape compensates for fabrication irregularities. However, the soft liner is not as durable and long lasting as all hard acrylic. Indirect restorations also require refinements. It is difficult to locate areas of distortions and most dentists allow up to an hour of office time to adjust and cement a crown.

The most difficult adjustment for the crown is the interproximal adjustment. This adjustment is tedious because it must be done slowly and incrementally, least too much is removed. If too much crown material is removed, an open contact is created. And if an open contact is inadvertently created by the dentist, then the crown must go back to the lab, and the patient must be scheduled for another appointment to complete the process. This scenario occurs occasionally to all dentists. It does not build confidence or self esteem. It may be confusing to the patient, since they know that the dentist took accurate dental impressions.

Having indicated that the most laborious crown and bridge adjustment for the dentist is the interproximal contact adjustment, the most important adjustment, in the inventor's opinion, is the actual fit of the crown to the prepared tooth structure. If there is any irregularity, the crown will not fully seat. Or, if the crown does fully seat, a slightly open margin may exist; that is, an open space existing between the crown and tooth. The crown may adapt well for most of the circumference around its margin to the tooth preparation, however, in one portion of the circle of the margin, there is lack of adaptation. An open margin would allow, over time, recurrent decay. It could also cause a floss catch or it could cause gingival irritation. The crown may fit the die well, but does not fit the tooth. Thus, checking the fit of the crown to the tooth is a necessary step. And this internal fit of the crown to the tooth is best verified by a thick medium checker. Two examples of a thick medium checker would be FIT CHECKER, a product made by GC America, and DISCLOSING WAX, made by Kerr Manufacturing. The thick consistency of these products not only show where a point contact of interference may exist, preventing full seating, they also show how far off the internal surface areas are that are not touching. Experience dictates that if there is distortion, the crown is too small and tight to the prepared tooth. It is always too small and never too large. If the interproximal contacts are off, the vast majority of time the contacts are too tight.

Much frustration exists. For example, one attached article from LVI VISIONS, illustrates an experience of ill-fitting restorations. Of note, is that the inquiring dentist is not contacting the dental school, the laboratory, or the manufacturers. Although there is technical support available from authoritative sources, no information exists anywhere that allows a dentist to make consistently accurate indirect restorations.

Lecturer Damon Adams, D.D.S. writes in SPECTRUM DIALOGUE that the problems of impression dentistry has been around for many years and it is yet to be eliminated or reduced in any significant degree. His entire article, attached herein, is very pertinent to this disclosure. He addresses the real problems with impressions and gives thoughtful solutions. It is clear that he, as all dental researchers, is unaware of the dental stone distortion. He associates the problems of ill fitting restorations with impression distortion. The attached article from DENTALTOWN MAGAZINE, shares a common experience; that the cast partial denture framework may fit the stone cast and not fit the mouth. The attached ad from a dental lab shows ill-fitting restorations could cost a dentist substantial income.

My personal dental professor/preceptor from dental school sought out the very best students after they had graduated. She wanted these gifted students to do her own dentistry. She related to me that she was never satisfied with any restoration.

I share the above information to point out that a very real problem exists in dentistry. Experts attempt their best for answers, but these are never complete, and indeed, can be misleading. The expert dentist responds in the LVI VISIONS article that the problem, of ill-fitting crowns, lies with the impression technique.

In CONTEMPORARY ESTHETICS AND RESTORATIVE PRACTICE, the author suggests that hydrocolloid impression materials avail superior dimensional accuracy.

In DENTISTRY TODAY, the author implies rigid materials in metal trays are most accurate. He also indicates that quadrant trays allow damaging movement by the patient's cheek and tongue and this unwanted movement will cause distortion.

Another dentist shares, in INSIDE DENTISTRY, that impressions should be removed with a rapid pull. He also feels that the shrinkage of the impression material should be offset by an equal expansion of dental stone. He comments that addition silicone materials give the lowest shrinkage.

The fact that experts give varying reasons for the problems proves first of all that the problems really do exist. This reality of ill-fitting restorations is best summarized by Sharp, et al. in U.S. Pat. No. 5,911,580. In column 1, they describe the normal process of a dentist usually having to adjust an appliance. They write of the possibility that the final outcome may be that the appliance can not be adjusted adequately. The appliance sometimes needs to be remade.

Prior art shows that there have been many attempts to identify the cause of ill-fitting crowns. In U.S. Pat. Nos. 6,045,359, 5,478,235, and 7,021,929 inventors modify dental impression tray designs. They hope to achieve better bonding between the impression material and the tray. Although it is true, that a bond is needed to the tray, any severe defect such as tray separation would create a gross discrepancy in fit of final appliance. These separations are usually evident to the practitioner at the time of tray removal from the oral cavity. Impression tray design alone has never made a further refinement in the fit of our dental castings.

Inventors of prior art have attempted to create new impression materials to increase accuracy. In U.S. Pat. Nos. 6,861,457 and 5,907,002 Kamohara recognizes the inaccuracies associated with polyether impression material. He further isolates the problems of inaccuracy to impression tray removal. He feels that recovery from deformation applied during time to remove the impression causes dimensional instability. He feels the solution for ill-fitting crowns is a more accurate impression material. With all of the impression materials on the market today, including the new ones introduced by Kamohara, there is very little improvement in the reliability of indirect dentistry.

Some inventors believe the inaccuracies occur after the impression is completed. They feel distortion occurs in the modeling process. This is the case with inventor Singer in U.S. Pat. No. 6,149,426. He states that inaccuracies occur with the master cast. His prior art confirms that the prosthetic must be fitted and corrected several times before it becomes accurate. He further feels that the problem, however, exists in the plastic trays used in the profession as well as subsequent modeling of the impression. His invention does not yield a better fitting crown.

Some inventors attribute a smooth stone cast and easy separation of the set cast from the impression as indications of accuracy. This is the case in two U.S. Pat. Nos. 5,907,002 and 3,620,778. Present day dental casts are distorted due primarily to shrinkage. Any reference to easy separation of dental cast would have this inventor immediately equate the observation to dental cast shrinkage. Most casts today exhibit distortion due to shrinkage.

In June 2006 issue of the JOURNAL OF THE AMERICAN DENTAL ASSOCIATION, Carl J. Drago, D.D.S. stated that the long term success of crowns and fixed partial dentures on natural teeth and implants depends on several variables: accuracy of fit between castings and abutments and teeth, impression materials and techniques, manipulation of dental stone, and casting and finishing processes.

The only problem with Drago's observations is that, heretofore, no one has come up with the correct combination to produce accurate castings.

PHILLIPS' SCIENCE OF DENTAL MATERIALS, edited by Kenneth J. Anusavice, PhD, DMD., states that if a partially set impression material is seated, it will be compressed elastically. Once the material has set completely, and the tray removed, then there will be a certain amount of spring back. This would cause the impression to produce smaller dies. The crowns made from such distorted dies would be tight to the tooth. Although this theory offers some limited insight, the persistent problems still cause frustration.

Die spacer may be applied to the die to compensate for shrinkage inherent to the system of modeling. However, stone die distortion occurs in random places and also to varying degrees. The deformation is unpredictable in regard to location as well as degree. So, if there is distortion, no amount of die spacer will compensate.

Also, die spacer can not be extended the entire length of the die. Spacer is not used to cover the margin. Die spacer on the margin of the die will cause an open margin of the crown preventing close adaptation to the tooth structure.

Removable appliances are also problematic. When the fit of new dentures is verified by the dentist, the buccal flanges tend to be tight along the outside alveolar ridge. The palatal area is usually distorted, so that space may exist between the palate and the internal surface of the denture. Removable appliances are also adjusted using a thick film medium. Dentists, using present day technology, trudge through the process of delivering the less than perfect indirect restorations, believing this is as good as it gets. They feel that the errors we see are a reflection of the limitations of the accuracies of all the systems involved. Professionals, familiar to the art, believe variables occur cumulatively with impression distortion, setting of dental stone, waxing techniques, investing and casting, and firing porcelain.

Appreciatively, after time is spent adjusting present day crowns, some crowns will fit very near perfect. However, some crowns can never be adjusted well enough for clinical acceptance. Crowns that are distorted beyond clinical acceptance become remakes.

Remakes are a disappointment to dentist as well as the patient. One dental laboratory owner admits his remake rate is 11%.

Realistically, most crown accuracy probably falls into a nebulous area. The crown is adjusted as well as possible, but the dentist is forced to accept slight imperfection. This is necessary. Taking another impression does not guarantee a better result.

As one experienced dentist related to me: he simply gave up. He gave up trying to troubleshoot the error.

One dentist feels the error occurs when the technician fits the crown to the model. In so doing, he feels that the technician inadvertently removes a small amount of stone when the prosthetic crown is tried on and off repeatedly from the die. This wearing away produces a crown that is too wide.

Another dentist stated that she feels the error exists in her impression; that the impression distorts in some way. She also feels that in some cases, the fault lies with the technician, so she'll change dental labs.

The experienced dentists usually do not share comment on outcome of prosthetic dentistry. And due to this reticence, dental technicians at the laboratories feel that the experienced dentist has certain skills and techniques that he has acquired over his career to enable him to make perfect crowns. In fact, many dental technicians are unaware of ill fitting restorations. Perhaps the more experienced dentist is more efficient at adjusting crowns, but his crowns fit no better than anyone else's. The inexperienced dentists are always trying to troubleshoot the problem, yet, in fact, experienced dentists learn that no solution exists.

There are so many steps in the dentist office, along with an equally complex arrangement in the laboratory, that dentists believe the solution for ill-fitting prosthetics, can never be achieved.

Distortion associated with indirect dentistry is the primary reason for growth of the use of CAD/CAM in dentistry. In U.S. Pat. No. 6,152,731 Jordan et al. describe how dentistry is moving toward digital images. Although digital images are an improvement in efficiency, cost is certainly a factor. And as far as overall accuracy, the concepts of the enclosed disclosure produce results that rival those of the CAD/CAM systems.

Partly because of the limitations of present day technology of impressions, we are now turning toward a digital world of CAD/CAM indirect restorations. These machines are very expensive, and still have limitations. They can not make a custom shaded crown, which may be necessary to match a single maxillary anterior tooth. This is a serious drawback in today's esthetic conscious world. And the CAD/CAM machines can not make dentures, partials, nightguard, or bleaching trays.

In the area of prosthetic eye fabrication, delivery and patient tolerance is also a problem. Due to distortions of shape from processing, eye wearers must return for adjustments to the prosthesis, just as denture patients return to the dentist.

Hearing aids made from impressions are no different. Inaccuracies are a problem. A certain percentage of dentures, artificial eyes, and hearing aids are returned to the laboratories for remakes.

There are many professions that have borrowed from the impression techniques of dentistry. Along with this borrowed technology came the problems of distortions; distortions that were never fully addressed in dentistry. No inventor or researcher has ever isolated the cause of distortion associated with indirect dentistry.

The advantage of this disclosure is to avail accurate indirect dental restorations, hearing aids, and artificial eyes.

### SUMMARY OF THE INVENTION

A dental impression is procured in a manner familiar to the dentist. This inventor has found no specific correlation to any specific type of impression material, or to any specific style of tray design, that effects final outcome.

This set impression is placed aside for use in the dental lab.

The impression surface is covered with a thin wet mix of dental stone to form a layer or veneer.

The stone lined impression is set aside for a complete cure of stone.

Last, the dental impression is filled to completion with wet, mixed dental stone in the traditional way.

### DETAILS OF THE PREFERRED EMBODIMENT

A very thin layer of mixed, wet dental stone is first applied to the internal or tissue side of the dental impression. This may be accomplished in different ways. The thin layer can be deposited directly by use of a syringe, filled with wet mixed dental stone. It may be desirable to utilize a fine tip syringe, such as a MONOJECT 412, manufactured by Tyco International. An even finer application can be achieved by use of AccuDose NeedleTubes, manufactured by Centrix Incorporation. Small bore syringe tips provide for a direct controlled deposit of wet mixed dental stone directly into the tooth prep, as well as adjacent teeth, of the dental impression. This more precise deposit of the wet stone will give a more uniform coating thickness to form a veneer or layer.

A fine spray of wet mixed stone could be applied with an air brush or spray applicator. This technique is already used to apply certain ceramic finishes, such as opaque layer of porcelain over metal or ceramic copings.

Alternatively, the technician may also chose to vibrate a portion of wet mixed stone into the impression and then immediately invert the impression and vibrate to expel all but a very thin veneer of wet stone, thereby, coating the internal surface of the impression.

This gypsum coated impression is set aside for complete curing of the dihydrate calcium sulphate dental stone before the subsequent addition of more dental stone to finalize the impression pour.

### CONCLUSIONS, RAMIFICATIONS AND SCOPE OF INVENTION

Implementation of this discovered technique enables dental impressions to yield more accurate casts than present day technology.

This distinctive invention advances dental restorations beyond present art and this invention improves the overall standard of care.

Accuracy, to this level, has never been appreciated in dentistry. This disclosed technique will add normalcy and stability to a process that, heretofore, is far from predictable.

To be able to reduce procedural time significantly is not to be understated. This technique will not only cut delivery time for the dentist in half, it produces a better product. Patients will enjoy restorations that will feel better and last longer.

Benefiting from new, improved accuracy, we can now further our studies and discoveries. For instance, numerous studies have been published comparing accuracy of various techniques and materials in use today. One notable experiment was to compare the accuracy of various impression materials after immersion into cold sterilizing solutions. Many of these studies were done in the early 1990s. It was determined that polyether was very likely to distort from gluteraldehyde sterilization.

Other studies determine how long an impression is dimensionally stable in the laboratory before pouring. There have also been studies comparing die stone dimensional accuracy when used in conjunction with different impression materials. Other studies examine powder/water ratios of die stone, effect of humidity on both impression materials and die stones, and effects of vacuum. Countless other studies for accuracy in dentistry are ongoing.

The inventor feels that data from studies, do not consider the effects described at length in this application. In short, much of the data assembled from studies of indirect dentistry may not be accurate.

It is further recognized by the inventor, that an extremely thin deposition of the wet stone would be similar to a thin coating of dihydrate calcium sulphate. This would serve as a setting stone catalyst and would enhance application Ser. No. 11/878,337.

The concept of the present invention is an initial layering significantly thick enough to maintain its own shape and accept additional layering of wet stone without distortion. The thickness of this initial layering could vary from barely perceptible to several millimeters thick, depending on laboratory technician's preferences with various types of impression materials and stone combinations.

The concept of additional layering to whole casts is not new to the dental profession. Layering is done for strength and support. There are many instances when stone additions are made incrementally as in mounting a cast, or layering to build a thicker and stronger model, or pouring a base.

However, the accuracy of the present invention over present day technique is obtained by the setting stability of a relatively thin coping. This is a unique concept. The thin coping or veneer is more accurate than a single, entire cast pour. The entire cast pour would allow shrinkage of stone to occur in areas replicating prepared teeth and this associated shrinkage will affect accuracy. This is the origin of indirect dentistry inaccuracies experienced today. The core of stone completely filling a tooth preparation impression is vastly more subject to stone setting shrinkage inaccuracies than a relatively thin coping pour of dental stone. Shrinkage in a filled tooth prep must be compensated in the peripheral borders, creating a smaller die. That is, shrinkage of stone will pull stone toward the center core of stone. However, shrinkage within a thin coping is compensated for. The free exposed surface of stone will distort and shrink, while maintaining integrity to the impression surface. The subsequent contiguous pour or more stone will set first closest to the set coping, forming a stratified set as described in application Ser. No. 11/878,337.

Very significant also, is to allow the dental stone veneer to completely cure, before the addition of more mixed wet dental stone.

Many researchers feel the stone is set after an hour or so, as stated by directives of stone manufacturers. PHILLIPS' SCIENCE OF DENTAL MATERIALS states that the time span from the start of mixing stone to loss of gloss from the surface of the mix may be only about fifteen minutes. The stone is ready to use in as little as thirty minutes. In use today, most technicians feel that one hour is sufficient stone setting time before the cast can be used. It is the experience of the inventor that more time is needed for a complete cure. A complete cure will resist the distortion caused by a subsequent juxtaposed stone pour. For instance, if the poured stone veneer is allowed to cure overnight, a subsequent pour into contact to this veneer will not alter the shape of the stone veneer. If the veneer is allowed to harden for only an hour, then a juxtaposed wet mix of stone has the potential to alter the shape of the stone veneer. This occurs even though the stone veneer is apparently set and seems hard when touched.

The concept of perfect fitting restorations is incomprehensible to expert professionals. Consider that there are over one hundred thousand dentists in this the United States alone, who collectively are fabricating over twenty million crowns a year. Discovering the necessary techniques to accomplish this level of accuracy has been elusive.

The article written by Eric K. Curtis, D.D.S. demonstrates a growing interest to incorporate a pay-for-performance value system in dentistry. That is, higher quality dentistry, which is better for the patient and takes more time, effort, and dedication on the part of the dentist, would deserve a higher fee than the norm. The technique herein described would provide this value.

This unique disclosure is long overdue. The profession has accepted status quo level of accuracy in dentistry for a very long time.

This invention has remained undiscovered because: A.) there are many complex procedures carried out by both laboratory technician and dentist to fabricate an indirect restoration, B.) no individual has learned techniques to compensate for and offset the distortions of setting stone, C.) the damage of a contiguous stone pour against an uncured previous stone pour is unknown, D.) the elevated results from the disclosure herein have never been achieved in dentistry, many dental professional experts believe accuracy at this new disclosed level would be unachievable and, E.) accuracy can only be achieved by the combination of two undiscovered concepts applied concomitantly, 1.) the concept of a stratified set of dental stone and, 2.) the concept of several hours long set time before modeling with a stone cast, instead of present day one hour set time before modeling.

1. An improved method for producing a stone dental cast comprising the steps of: a. placing a thin wet, mixed dental stone layer onto dental impression surface, said mixed dental stone layer being sufficiently thin to capture detail of said impression surface and said mixed dental stone layer being sufficiently thin to maintain open core of impression cavity, b. allowing complete cure of said mixed dental stone layer, then, c. pouring sufficiently more mixed stone to make a complete cast.

2. An improved method of forming a stone dental cast comprising the steps of:
a) attaching dental stone veneer onto internal surface of dental impression, said dental stone veneer being sufficiently thin to capture said dental impression detail and said dental stone veneer being sufficiently thin to make a coreless fill of said dental impression;
b) allowing full cure of said dental stone veneer;
c) pouring wet, mixed dental stone into said dental impression, and into contact with said dental stone veneer, whereby, a dimensionally exact cast is produced.

3. An improved method for producing a stone cast comprising the step of: applying a stone layer to the internal surface of impression material, whereas said stone layer is sufficiently thin to capture detail of said impression material, and further whereas said stone layer is sufficiently thin to maintain open chamber of impression cavity, and allowing full set of said stone layer, whereby adding wet, mixed stone juxtaposed to said impression material and said stone layer, sets to form an accurate cast.

4. A method according to claim 3, wherein said impression material replicates oral structure.

5. A method according to claim 3, wherein said impression material replicates an ear canal.

6. A method according to claim 3, wherein said impression material replicates an eye structure.
